SEQUENCE LISTING (12 sequences)

SEQ ID NO: 1 - DNA, 24 bp, Artificial Sequence
  Description of Artificial Sequence Synthetic primer
ctgatcgaatggctgccaggctcc

SEQ ID NO: 2 - DNA, 24 bp, Artificial Sequence
  Description of Artificial Sequence Synthetic primer
caaccagacgatagttatcacgca

SEQ ID NO: 3 - DNA, 25 bp, Artificial Sequence
  Description of Artificial Sequence Synthetic primer
cctgatcgcactgaatatcgtactg

SEQ ID NO: 4 - DNA, 23 bp, Artificial Sequence
  Description of Artificial Sequence Synthetic primer
gaccatcaccaatggtcagcagg

SEQ ID NO: 5 - DNA, 24 bp, Artificial Sequence
  Description of Artificial Sequence Synthetic primer
ctcagacgggatttgttaggcacg

SEQ ID NO: 6 - DNA, 23 bp, Artificial Sequence
  Description of Artificial Sequence Synthetic primer
gatcttggagcattcccacaacc

SEQ ID NO: 7 - DNA, 23 bp, Artificial Sequence
  Description of Artificial Sequence Synthetic primer
agcgccgcggtacgggcgataaa

SEQ ID NO: 8 - DNA, 24 bp, Artificial Sequence
  Description of Artificial Sequence Synthetic primer
atcatcgtcattaatgcctaacgt

SEQ ID NO: 9 - DNA, 20 bp, Artificial Sequence
  Description of Artificial Sequence Synthetic primer
tgtatgttgatactaaatcc

SEQ ID NO: 10 - DNA, 18 bp, Artificial Sequence
  Description of Artificial Sequence Synthetic primer
ctgtcatgcagtaaccag

SEQ ID NO: 11 - DNA, 21 bp, Artificial Sequence
  Description of Artificial Sequence Synthetic primer
atgaatatgaatcagaccacc

SEQ ID NO: 12 - DNA, 21 bp, Artificial Sequence
  Description of Artificial Sequence Synthetic primer
ggcgtatagtcggcggttttc

(19) United States
(12) Patent Application Publication
(10) Pub. No.: US 2007/0218468 A1
(43) Pub. Date: Sep. 20, 2007

(54) Method for detecting the presence of water born pathogens and indicator microorganism

(76) Inventors: Hemant Jyotiswarup Purohit, Nagpur (IN); Atya Kapley, Nagpur (IN); Dhananjay Vasant Raje, Indore (IN); Sukumar Devotta, Nagpur (IN)

Correspondence Address: EDWARDS ANGELL PALMER & DODGE LLP, P.O. BOX 55874, BOSTON, MA, 02205, US

(21) Appl. No.: 11/506,916

(22) Filed: Aug. 18, 2006

(30) Foreign Application Priority Data
Mar. 20, 2006 (IN) 0729/DEL/2006

(51) Int. Cl. C12Q 1/68; C12P 19/34

(52) U.S. Cl. 435/6; 435/91.2

(57) ABSTRACT

The present invention relates to a method for detecting the presence of water born pathogens and indicator microorganism including bacteria from water sample by selecting the target gene carried in template DNA by amplifying the target DNA using specific primers with biotinylated tag consist of all or a substantial part of 5′-CTGATCGAATGGCTGCCAGGCTCC-3′ and 5′-CAACCAGACGATAGTTATCACGCA-3′ and taq DNA polymerase to get desired biotinylated tagged probe followed by hybridization of biotinylated tagged probe with target gene in template DNA followed by enzyme coupled reaction.

### FIELD OF THE INVENTION

The present invention relates to a method for detecting the presence of water born pathogens and indicator microorganism including bacteria.

Particularly, it relates to a method for detecting the presence of water born pathogens and indicator microorganism including bacteria by using specific biotinylated primers consist of all or a substantial part of 5′-CTGATCGAATGGCTGCCAGGCTCC-3′ (SEQ ID NO: 1) and 5′-CAACCAGACGATAGTTATCACGCA-3′ (SEQ ID NO: 2).

### BACKGROUND AND PRIOR ART OF THE INVENTION

Diseases caused by water borne pathogens are of concern all over the world. In developing countries, the lack of proper sanitation leads to contamination of clean potable water. The intensity of the problem was reported way back in 1982, with over 250 million reported cases of waterborne disease and more than 10 million deaths annually (Snyder and Merson, 1982). The World Health Organization (WHO) report of March 2001 also stated that more than 3 million people die annually from water-related diseases. When other sources of diarrheal disease are taken into consideration, the figures are even more staggering, with these diseases claiming the lives of over 5 million children per year in developing countries (Hale, 1991). However, routine monitoring of water samples for possible contamination is practically not feasible. An accepted alternative is to look for indicator bacteria associated with pathogens of fecal origin (Nelis and Van Poucke, 2000).

Reference may be made to Rompre et al., 2002 wherein they have used coliform group and more specifically E.coli, as an indicator of microbial water quality. Over a period of time the approaches towards analysis have changed but the coliforms still remained as markers in drinking water safety measures. Since E.coli always originates from the intestine of humans and warm-blooded animals, it is the preferred indicator bacteria for monitoring fecal contamination of water samples. The presence of E.coli in any water sample is often correlated to the risk of numerous diseases (Chudzik et al., 2003). The conventional method for detection of bacterial contamination in water samples is time consuming and involves classical culture based methods and biochemical tests that is not acceptable in epidemic situations. However, depending on environmental stresses, like oxidative stress, bacteria enter into a viable but non-culturable form. Such forms cannot be detected by conventional tests (Knight et al. 1990; Baudart et al. 2002).

Various methods for detection of E.coli in water samples have been reported. Reference may be made to Ley, et al. (U.S. Pat. No. 4,923,804) wherein a method specific to determining and enumerating E.coli in water samples or sewerage specimens was developed. The method comprises adding to a cultured specimen, a chromogenic reagent which when subjected to in situ E.coli beta.-glucuronidase activity produces clearly defined vivid colorization of any individual colonies derived from E.coli cells in the test specimen. Reference may be made to Roll (U.S. Pat. No. 5,627,275) wherein the details of growth medium dependent water quality monitoring and its limitation have been described. Furthermore, reference may be made to Armstrong et al. (U.S. Pat. No. 5,858,698) wherein they provided therapeutic and diagnostic methods for treatment and detection of enteropathogenic E.coli (EPEC) enteric infections. Brenner et al. (U.S. Pat. No. 6,306,621) came out with an improved method for detection of E.coli comprising a broth containing an ingredient that will encourage growth and repair of injured coliforms.

However, the drawbacks of these methods are like:
tediousness of the method
maintaining viability of bacteria between the time of collection and enumeration lack of growth of viable but non-culturable bacteria, such as those stressed by chemicals in the water
failure to cultivate all living cells of interest, time (days) required for detection and confirmation of enteric bacteria
lack of specificity for detection of true fecal coliforms such as E.Coli
failure to distinguish living from dead cells using direct microscopic counts and
wrong identification of organisms due to antigenic cross reactivity using serological procedures.

In addition, the methods to detect these pathogens were initially designed for clinical, rather than environmental samples. Since the ecological niche occupied by clinical samples differs from that of environmental isolates it is not necessary that the same protocol can be applied to samples collected from either niches. Hence, there is a need to develop a specific protocol for detection of bacteria from environmental niches.

### OBJECTS OF THE INVENTION

The main object of the present invention is to provide a method for detecting the presence of water born pathogens and indicator microorganism including bacteria from water sample.

Another object of the present invention is to provide a method for detecting the presence of water born pathogens and indicator microorganism including bacteria from water sample by using biotinylated tagged specific primers consist of all or a substantial part of 5′-CTGATCGAATGGCTGCCAGGCTCC-3′ (SEQ ID NO: 1) and 5′-CAACCAGACGATAGTTATCACGCA-3′ (SEQ ID NO: 2).

### SUMMARY OF THE INVENTION

The present invention deals with a method for detecting the presence of water born pathogens and indicator microorganism including bacteria from water sample by selecting the target gene carried in template DNA by amplifying the target DNA using specific primers with biotinylated tag consist of all or a substantial part of 5′-CTGATCGAATGGCTGCCAGGCTCC-3′ (SEQ ID NO: 1) and 5′-CAACCAGACGATAGTTATCACGCA-3′ (SEQ ID NO: 2) and taq DNA polymerase to get desired biotinylated tagged probe followed by hybridizing the biotinylated tagged probe with target gene in template DNA followed by enzyme coupled reaction.

### BRIEF DESCRIPTION OF THE DRAWINGS

The following Detailed Description, given by way of example, but not intended to limit the invention to specific embodiments described, may be understood in conjunction with the accompanying drawings, in which:

FIG. 1 shows a glass slide protocol where the E.coli template has been mobilized on activated glass surface and assayed as been optimized for nylon membrane.

FIG. 2 shows an extension of glass-slide immobilization protocol where the template is immobilized on nylon membrane that can be easily detected by the presence of blue color.

### DETAILED DESCRIPTION OF THE INVENTION

Accordingly, the present invention provides a method for detecting the presence of water born pathogens and indicator microorganism including bacteria from water sample, wherein the said method comprising the steps of:
a) providing water sample and concentrating into suitably sized concentrate of containing target indicator microorganisms in said water sample;
b) isolating the template DNA from target cells obtained from step (a);
c) selecting the target gene carried in said template DNA obtained from step (b) by amplifying the target DNA by using appropriate biotinylated tagged primers and taq DNA polymerase to get desired biotinylated tagged probe containing target gene;
d) hybridizing the biotinylated tagged probe obtained from step (c) with target gene present in the said template DNA followed by enzyme coupled reaction as resulting by change in color wherein blue color indicates the presence of indicator microorganism in the test sample and absence of blue color indicates the absence of said microorganism in the test sample.

In an embodiment of the present invention, the water sample is collected from a polluted source selected from any contaminated drinking water.

In another embodiment of the present invention, the target cell may be selected from the group of enteric bacteria such as E.coli, Salmonella, Vibrio etc.

Further, in another embodiment of the present invention, the target gene may be selected from lamb gene of E.coli, InvA gene, PhoE gene, SpvA gene and SpvB gene of Salmonella and Ctx gene of Vibrio etc.

In yet another embodiment of the present invention, the following primer sequences are used for detection of presence of enteric pathogens:
a) Upper: 5′-CTGATCGAATGGCTGCCAGGCTCC-3′ (SEQ ID NO: 1) and Lower: 5′-CAACCAGACGATAGTTATCACGCA-3 (SEQ ID NO: 2) for detection of E-Coli based on target gene lamB;
b) Upper: 5′-CCTGATCGCACTGAATATCGTACTG-3′ (SEQ ID NO: 3) and Lower: 5′-GACCATCACCAATGGTCAGCAGG-3′ (SEQ ID NO: 4) for detection of Salmonella based on target gene InvA;
c) Upper: 5′-CTCAGACGGGATTTGTTAGGCACG-3′ (SEQ ID NO: 5 and Lower: 5′-GATCTTGGAGCATTCCCACAACC-3′ (SEQ ID NO: 6) for detection of Vibrio based on target gene CtxA;
d) Upper: 5′-AGCGCCGCGGTACGGGCGATAAA-3′ (SEQ ID NO: 7) and Lower: 5′-ATCATCGTCATTAATGCCTAACGT-3′ (SEQ ID NO: 8) for detection of Salmonella based on target gene phoE;
e) Upper: 5′-TGTATGTTGATACTAAATCC-3′ (SEQ ID NO: 9) and Lower: 5′-CTGTCATGCAGTAACCAG-3′ (SEQ ID NO: 10) for detection of Salmonella based on target gene spvA;
f) Upper: 5′-ATGAATATGAATCAGACCACC-3′ (SEQ ID NO: 11) and Lower: 5′-GGCGTATAGTCGGCGGTTTTC-3′ (SEQ ID NO: 12) for detection of Salmonella based on target gene spvB;

In still another embodiment of the present invention, the said template DNA is isolated from target cell by dipping the disc in 0.5N NaOH solution on a cling film for 2-5 minutes followed by similar treatment using Tris solution wherein the ratio of NaOH solution and Tris solution used 1:1.

In still another embodiment of the present invention, the template DNA used is isolated from commercially available strain Escherichia strain ATCC 35150.

In still another embodiment of the present invention, the target gene used is lamB gene, present in all species of the genera Escherichia.

### In still another embodiment of the present invention, the primer sequences used consist of all or a substantial part of 5′-CTGATCGAATGGCTGCCAGGCTCC-3′ (SEQ ID NO: 1) and 5′-CAACCAGACGATAGTTATCACGCA-3′(SEQ ID NO: 2).

In still another embodiment of the present invention, the generated biotinylated tagged probe has a size of approximately 309 bp.

In still another embodiment of the present invention, the amplified sequence of the lamB gene generated using optimized conditions for pre-hybridization and hybridization of biotinylated tagged probe to the target gene present in the said recovered target DNA.

In still another embodiment of the present invention, the pre-hybridazation is carried out with 200 μl-220 μl of hybridization buffer for 15 minutes at room temperature followed by hybridization wherein denatured lamB probe in sterilized distilled water is added in hybridization buffer.

In still another embodiment of the present invention, the denaturation of lamB probe is carried out at 95 degree C. for 5-6 minutes in water bath.

In still another embodiment of the present invention, the lamB probe used is in concentration about 25 ng.

In still another embodiment of the present invention, the disc is transferred in a fresh glass tube followed by washing it with 2X SSC and TBS.

In still another embodiment of the present invention, the non-specific signals is blocked by using blocking solution of 1.5% BSA in TBS at 55 degree C. for 10-15 minutes

In still another embodiment of the present invention, the signal is generated by diluting Streptavidin-Alkaline phosphatase in the ratio ranging from 1:2000 fold in BSA in TBS at room temperature with biotinylated-hybridized probe.

In still another embodiment of the present invention, excess and non-specifically bound Streptavidin-Alkaline phosphatase is removed by washing with Tris-NaCl SDS buffer at pH-8.0.

In still another embodiment of the present invention, the signal amplification has been devised with substrate and amplifier for Streptavidin-Alkaline phosphatase as a powder is freshly mixed separately with dilution buffer for a period of 15 minutes.

In still another embodiment of the present invention, the substrate used is reduced nicotinamide adenine dinucleotide phosphate.

In still another embodiment of the present invention, the amplifier used is enzymes—alcohol dehydrogenase and diaphorase

In still another embodiment of the present invention, the blue color is developed which indicates the presence of water born pathogens and indicator microorganism including bacteria wherein the said color is stabilized by using 0.5N KOH and acetone in the ratio 5:3.

In still another embodiment of the present invention, blue color is developed immediately by adding KOH and acetone.

### In still another embodiment of the present invention, an enzyme-coupled reaction with hybridization increases the sensitivity of the detection protocol.

The rapidity, feasibility and cost effectiveness of method points to benefits in drinking water analysis, particularly in epidemic situations and thus facilitates improved public health management

Molecular tools as water monitoring over the conventional tools (Kapley et al., 2000; Kapley et al., 2001; Purohit and Kapley, 2002) has been used to solve these kind of problem. Earlier, Abramowitz (1996) reviewed the use of molecular tools in diagnostic applications. The Polymerase Chain Reaction (PCR) is one such established molecular technique, which reliably identifies a segment of DNA and it uses a set of specific sub-sequences to amplify target segment DNA from a mixed template population. The efficiency of detection can be increased by designing a duplex PCR, wherein two pathogens/loci can be detected in a single reaction (Kapley et al., 2000a; Kapley et al., 2001a). The multi-step thermo cycling program can further add to the specificity of analysis as demonstrated for the simultaneous detection of three target loci in a single reaction (Kapley et al., 2000). Based on PCR technique, the detection and monitoring of water borne pathogen has been extensively explored. Amongst the pathogens, E.coli, Salmonella, and Vibrio are the most reported ones. The primer sets used to monitor these pathogens by PCR have been listed in Table 1. Bej et al. (1990) have shown that E.coli, an indicator bacterium, can be monitored by amplifying a PCR product using the lamB gene. Salmonella could be monitored using the invA locus, which has also been demonstrated in river water samples. Both these loci encode for the bacteriophage specific surface proteins whereas, for monitoring Vibrio, a toxin encoding locus ctxA, has been used.

TABLE 1

Evaluated primers for most probable enteric bacteria observed
in drinking water

| | | | | |
|---|---|---|---|---|
| S. | Target | Target | | |
| No. | Loci | Organism | Primer Sequence | Reference |

| | | | | |
|---|---|---|---|---|
| 1. | Lamb | E.coli | Upper: | Kapley et al. |
| | | | 5′-CTGATCGAATGGCTGCCAGGCTCC-3′ | (2000); Bej et |
| | | | (SEQ ID NO: 1) | al. (1990) |
| | | | Lower: | |
| | | | 5′-CAACCAGACGATAGTTATCACGCA-3′ | |
| | | | (SEQ ID NO: 2) | |
| 2. | InvA | Salmonella | Upper: | Kapley et al. |
| | | | 5′-CCTGATCGCACTGAATATCGTACTG-3′ | (2000) |
| | | | (SEQ ID NO: 3) | |
| | | | Lower: | |
| | | | 5′-GACCATCACCAATGGTCAGCAGG-3′ | |
| | | | (SEQ ID NO: 4) | |
| 3 | CtxA | Vibrio | Upper: | Kapley et al. |
| | | | 5′-CTCAGACGGGATTTGTTAGGCACG-3′ | (2000) |
| | | | (SEQ ID NO: 5) | |
| | | | Lower: | |
| | | | 5′-GATCTTGGAGCATTCCCACAACC-3′ | |
| | | | (SEQ ID NO: 6) | |
| 4. | phoE | Salmonella | Upper: | Kapley et al. |
| | | | 5′-AGCGCCGCGGTACGGGCGATAAA-3′ | (2001) |
| | | | (SEQ ID NO: 7) | |
| | | | Lower: | |
| | | | 5′-ATCATCGTCATTAATGCCTAACGT-3′ | |
| | | | (SEQ ID NO: 8) | |
| 5. | spvA | Salmonella | Upper: | Kapley et al. |
| | | | 5′-TGTATGTTGATACTAAATCC-3′ | (2001) |
| | | | (SEQ ID NO: 9) | |
| | | | Lower: | |
| | | | 5′-CTGTCATGCAGTAACCAG-3′ | |
| | | | (SEQ ID NO: 10) | |
| 6. | spvB | Salmonella | Upper: | Kapley et al. |
| | | | 5′-ATGAATATGAATCAGACCACC-3′ | (2001) |
| | | | (SEQ ID NO: 11) | |
| | | | Lower: | |
| | | | 5′-GGCGTATAGTCGGCGGTTTTC-3′ | |
| | | | (SEQ ID NO: 12) | |

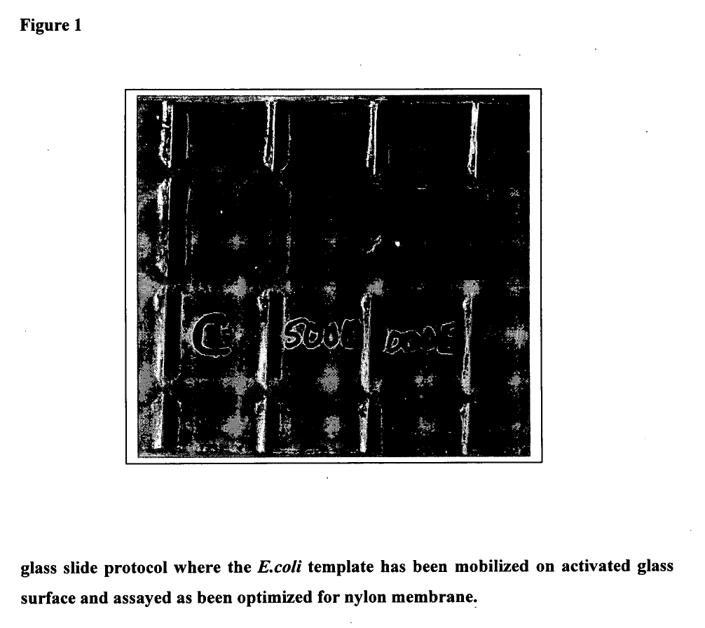

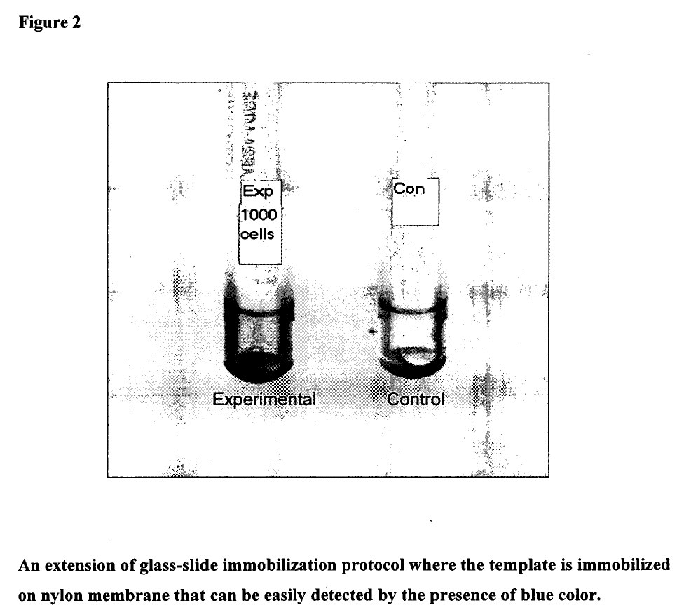

The present invention combines various techniques such as, development of gene probes using PCR, followed by detection using an enzyme-coupled assay. A rapid protocol has been established for hybridization followed by washing steps to ensure targeted binding of probe to selected indicator locus. The detection is carried out using color reaction where a color less substrate is converted to a pink colored product via a coupled redox reaction and stabilized under alkaline condition as blue end product. The present invention is illustrated in FIG. 1 to 2 of the drawings accompanying this specification. FIG. 1 represents the glass slide protocol where the E.coli template has been mobilized on activated glass surface and assayed as been optimized for nylon membrane. FIG. 2 represents an extension of glass-slide immobilization protocol where the template is immobilized on nylon membrane that can be easily detected by the presence of blue color.

The method uses a gene probe from the lamB locus that is specific to fecal E.coli (Kapley et al., 2000). The target locus was amplified by PCR and the amplified product was biotinylated to develop the gene probe. This probe was used in all the experimental protocols. The template (E.coli) was immobilized on a support and bacteria were lysed to release the DNA. Hybridization of the immobilized DNA with the probe, followed by the detection protocol, resulted in a colorimetric estimation protocol.

The protocol was optimized for the detection of pathogenic E.coli under different parametric conditions. The quantitative data shown below is the average of three independent experiments.

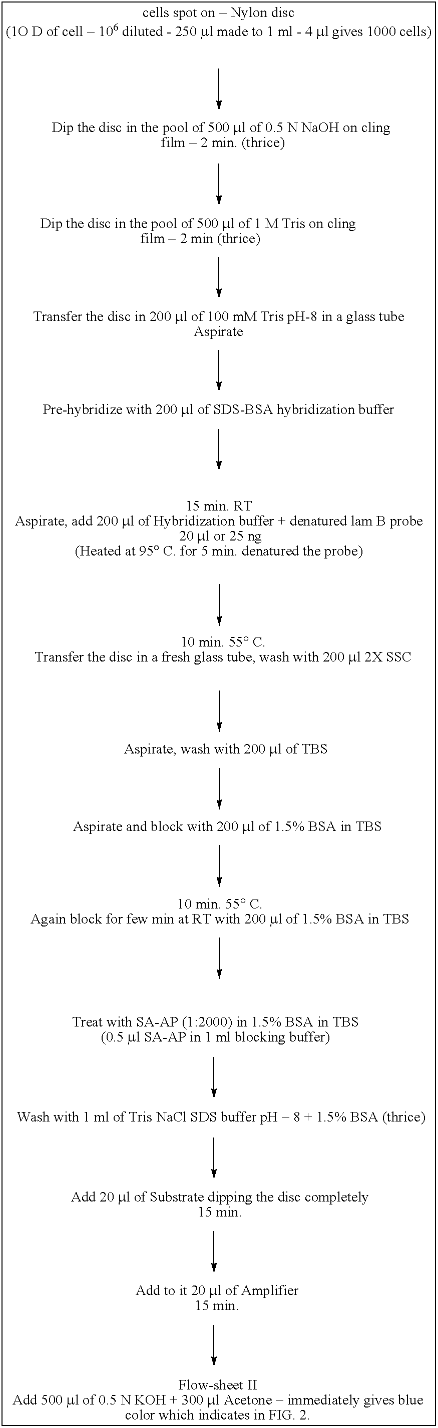

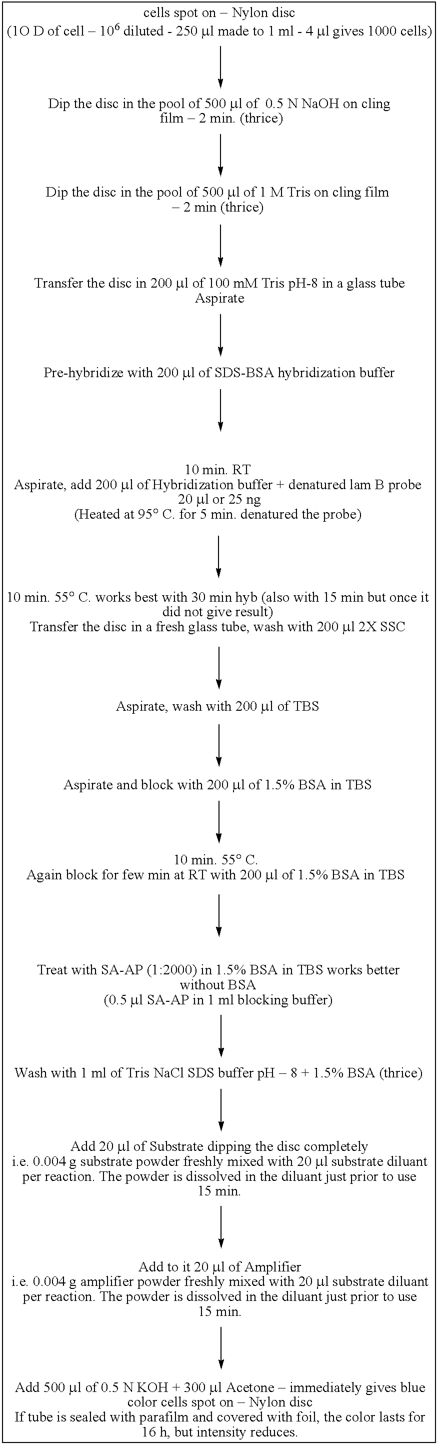

The following examples are given by way of illustration of the present invention and therefore should not be constructed to limit the scope of the present invention.

### EXAMPLE-1

### 96 Well Plate Protocol was Optimized for Detection of 1000 Cells:

Activation of the plate with TBS-SDS

Cells+DDW+NaOH—in eppendorff

Printing buffer+reaction mixture from the eppendorff

Incubate for 30 min

Wash twice with water

Block with hybridization buffer—30 min RT

Aspirate and add denatured probe (5, 10, 20 μl; 35 ng/μl) 5 min 95° C.

After this incubation put immediately on ice

Wash with 1×SSC pH 7

SAP 1:500 dil. RT for 30 min.

Wash twice with TBS-tween 20 buffer

Substrate (NADPH)—10 min in dark at RT

Amplifier—10 min in dark at RT

The well containing cell appears pink colored.

Following sets of variation in protocol tried at the selected steps:

1. 500 and 1000 cells, diluted NADPH substrate
No color change

2. 1000 cells, substrates tried ELISA BRL and 10 mM NADPH, probe (5,10,20 μl)
Dark pink color in BRL substrate and light pink in NADPH substrate.

3. Same as above except NADPH (100 mM)—(2,5,10 μl)
5 and 10 μl showed very light pink color but 2 μl didn't show any color.

4. 100, 500 1000 cells followed in the same way in original protocol as previous except detection of probe was carried out using the chemiluminiscent kit from Gibco BRL, USA.
Clear gradation in the blots of the X-ray film appeared as per the cell concentration as: 1000>500>100.

5a. The same process (as above in 4) was followed but substrate and amplifier from the ELISA kit, Gibco-BRL, was used for colorimetric analysis.
Results were not as clear as seen with the chemiluminiscent kit.

5b. The same protocol was followed but the nylon membrane was cut at the pencil marked circles indicating template position and protocol was followed in the eppendorff for colorimetric analysis and the reaction was stopped by adding 0.3M H₂SO₄. Template used in the reaction was bacterial cells and total DNA.
Total-DNA showed color gradation but no results were observed when cells were used as template.

6. The same procedure as done above was tried with different termination time of 5 and 15 min.
Total-DNA showed result but cells didn't show any gradation.

7. The same protocol was followed in duplicate—one with blocking and other without blocking (1.5% and 3% milk powder), with two different hybridization buffers, one with BSA and the other containing 5×Denhardt's solution.
hybridization buffer containing 3% BSA showed color gradation (1000<500<100). No results were observed in the other reactions.

8. The same procedure was repeated with 4 different concentration of milk powder (0.5%, 1%, 1.5% and 3%)
No color gradation in any case.
   With plastic 96 well plates as an option, the sensitivity of 1000 cells was observed. However, the results were not reproducible. Hence, the detection protocol was tested on different supports.

### EXAMPLE-2

### Nylon Membrane or FTA Filter as Immobilizing Matrix for Template:

Blotting and development procedure:

Nylon membrane strip was cut

E.coli Cells were incubated with 0.5N NaOH for 30 min at room temperature (RT) in eppendorff tubes

To this reaction mixture printing buffer was added

The reaction mixture was spotted on the nylon membrane

Template immobilization was done by UV cross linking

Wash membrane with 100 mM Tris pH 8.0

Pre hybridize for 10 min

Block with blocking agent

Denatured probe (5, 10, or 20 μl; 50 ng/μl) 5 min 95° C.

Aspirate and hybridize membrane bound template with the probe for 1 h at 60° C.

Wash the blot in a fresh tube with 2×SSC

1:1000 diluted SAP, in hybridization buffer for 10 min at RT

Wash with TBS

Cut the paper and put in the eppendorff

Treat it with substrate and amplifier

Observe pink coloration for colorimetric analysis

Following sets of variation in protocol tried at the selected steps:

1. 1000 cells, 1:1000 SAP, with and without blocking (3% milk powder)
No color difference.

2. The same protocol was repeated without using printing buffer and SAP dilution—1:500.
No result

3. Same procedure with different probe concentration—2, 4, 8 μl
Slight color difference appeared.

4. Same protocol was repeated on nylon membrane and FTA filter, concentration of probe was varied probe (2, 5, 10 μl)
No difference in the result

5. The blots were washed with 1M Tris pH 9 after UV cross linking of template
No color difference.

6. The dilution of SAP was changed to 1:2000 keeping all other factors constant
Color difference seen between control and E.coli template

### Few Changes were Done to this Protocol

After spotting the sample, the nylon membrane was incubated for 15 min. at 56° C. and the membrane was UV cross linked as usual

After addition of ELISA kit amplifier and incubation, to all the tubes 0.08N NaOH (300 μl) was added followed by acetone (500 μl)

Blue color develops after the addition of acetone

The color in the negative control tube fades to almost colorless however, the color in the tubes containing template E.coli remains dark blue.

7. Same procedure as above with SAP dilution—1:2000 and 1:5000, probe 10 μl
Color gradation appeared in 1:2000 dilution but not in 1:5000 diluted SAP.

8. 1000 cells, UV cross-linked, 1:2000 SA-AP, 10 μl probe Substrate (20 μl) for 10 min, pulse and take the supernatant in fresh tube add amplifier (20 μl) for 30 min and 0.08N NaOH
No result

### Same Procedure of Probe Hybridization at 55° C. with Some More Changes

Instead of spotting the reaction mixture on the membrane, the cell were directly spotted on the nylon disc

It was then pooled in 0.5N NaOH for 2 min.—soaked on filter paper

In a pool of 1M tris pH 7.5—2 min. at RT—soak it

This NaOH and tris treatment was repeated thrice

Dry at RT, then followed by the same previous protocol.

9. 1000 cells, SAP 1:2000 diluted substrate and Amplifier incubation time—10, 20. 30 min.
10 and 20 min incubated tubes showed color difference but not in 30 min tube.

10. The only change was in hybridization time with different concentration of probe
Probe—5 μl for 10 min and 1 h
   10 μl for 10 min. and 1 h

### The Same Incubation Time was Used for Blocking (3% BSA)

Different probe concentration for different time showed color Gradation observed, however, it was not uniform

11. Two different probe concentrations—10 and 50 μl, 10 min probe hybridization, rest all same.
10 μl probe showed color gradation between water and cell tube
No difference in the other tube.

12. 1000 cells, 1:2000 SAAP, substrate and amplifier (15 min. incubation) blocking done after probe hybridization wash and with TBS in blocking buffer, SDS/BSA hybridization buffer

Blocking agent with conc.—1.5%, 3% and 5% BSA in TBS

O D at 610 nm:—1.5%—0.1358
3%—0.1213
5%—0.0441

13. Same as above except amplifier with 15, 30, 60 min incubation time.

O D:—15 min—0.4269
30 min—0.0006
60 min—0.2858

14. Different blocking agents were tried—1.5% casein, BSA, tryptone, betaine in TBS

O D:—Betaine—0.0829
BSA—0.0552
Tryptone—0.0902
Casein—0.0642

15. BSA 1.5%, after amplifier incubation—0.25N and 0.5N, NaOH and KOH, fresh vial was taken after probe hybridization step

O D:—0.25N NaOH—0.0126
0.50N NaOH—0.3659
0.25N KOH—0.0346
0.50N KOH—0.494

### The Color Intensity of Blue Solution with KOH was Higher than NaOH.

16. 1000 cells, Probe 10 μl, in the color developing step 0.5N KOH was used in combination with acetone and DMF.

O D:—10 μl probe with acetone—0.6140
10 μl probe with DMF—0.0485

17. Different probe concentration—2.5, 5, 10, 15, all other parameters unchanged

O D:—2.5 μl Probe—0.0261
5 μl Probe—0.3370
10 μl Probe—0.4170
15 μl Probe—0.2818
20 μl Probe—0.5933

18. The above procedure was repeated again to test reproducibility

O D:—2.5 μl—0.0293
5 μl—0.3721
10 μl—0.3970
15 μl—0.4752
20 μl—0.5802

19. Different SAP concentration—1:500, 1:1000, 1:2000 and 1:5000, probe—10 μl

O D:—1:500 SAP—0.2868
1:1000 SAP—0.2871
1:2000 SAP—0.2788
1:5000 SAP—0.5039

20. With more dilutions of SAP—1:100, 1:500, 1:1000, 1:2000, 1:3000, 1:4000, 1:5000, 1:6000, 1:8000, 1:10000 using same probe concentration (10 μl)

O D:—1:100 SAP—0.0803
1:500 SAP—0.0935
1:1000 SAP—0.2112
1:2000 SAP—0.2788
1:3000 SAP—0.3039
1:4000 SAP—0.2788
1:5000 SAP—0.3069

With other dilutions no reproducible results obtained.

21. Probe concentration varied—20 μl and 30 μl
Very high background
   Based on the above-mentioned permutation and combinations the protocol was optimized on glass plate and also nylon membrane disc. In case of nylon disc the generated signal was extracted in 3 ml glass tube for semi-quantitative assay.

### EXAMPLE-3

### Hybridization Protocol for 1000 Target Cells of E.coli

Composition of the buffers used for this protocol:

1. Hybridization buffer: 1M Sodium phosphate buffer pH 7.2-250 ml [33.5 g Na₂HPO₄, 1 ml ortho-phosphoric acid (85% H₃PO₄)]
Add 1 ml 0.5M EDTA pH 8.0
Add 5 g BSA
14% SDS—250 ml.

2. 2×SSC (500 ml): NaCl—8.76 g
Trisodium citrate—4.11 g
DDW—400 ml.

3. TBS—Tween20: 1M Tris—25 ml
5M NaCl—7.5 ml
Tween 20—125 μl

4. Blocking buffer: 1.5% BSA—0.15 g BSA in 10 ml of TBS

5. Tris NaCl SDS: Tris (100 mM)—1.2114 g %

Buffer pH 8.0 NaCl (150 mM)—0.8766 g %
SDS—0.05%

### Advantages:

The main advantages of the present invention are:

Handling and preparation of larger number of sample could be carried out on site.

DNA extraction protocol is rapid and doesn't require any expensive chemicals.

The invention not only considers viable bacteria but also targets viable but non-culturable bacteria, such as those stressed by chemicals in the water and those lost their potential to grow even on the prescribed medium.

Total elimination of growth medium and cultivation steps for confirmation of enteric bacteria.

Gene probes are very specific for detection of true fecal coliforms such as E.coli and there is no wrong identification of organisms such as the approach used in serological procedures where antigenic cross reactivity lead to higher number of false positive/negative.

The coupled enzyme redox reaction enhances the sensitivity of the detection.

The method has been optimized specifically for lamB locus which eliminates the non-specific and false positive reaction to ensure the specificity of the detection method.

The method is cost effective with simplicity in sample preparation followed by detection protocol; at the same time doesn't require any sophisticated instrumentation or training for user.

### REFERENCES

### U.S. Patent Documents

1. Ley, A. N., et al., “Escherichia coli (E.coli) test method”, U.S. Pat. No. 4,923,804, May 1990.

2. Roll, B., “Detection and identification of enteric pathogens,” U.S. Pat. No. 5,627,275, May 1997.

3. Armstrong, G. D., et al., “Methods for detection of enteropathogenic E.coli, ” U.S. Pat. No. 5,858,698, January 1999.

4. Brenner, K. P. et al., “Membrane filter agar medium for simultaneous detection of total coliforms and E.coli.,” U.S. Pat. No. 6,306,621, October 2001.

### OTHER REFERENCES

5. Snyder, J. D. and Merson, M. H., “The magnitude of the global problem of acute diarrheal disease: a review of active surveillance data,” Bull. World Health Organ., 60: 605-613 (1982).

6. Hale, T. L., “Genetic basis of virulence in Shigella species,” Microbiol. Rev., 55: 206-224 (1991).

7. Nelis, H and Van Poucke, S., “Enzymatic detection of coliforms and Escherichia coli within 4 hours,” Water Air and Soil Pollution, 123: 43-52 (2000).

8. Rompre, A.,Servais, P., Baudart, J. & Marie-Renee de-Roubin, and Laurent, P., “Detection and enumeration of coliforms in drinking water: current methods and emerging approaches,” Journal of Microbiological Methods, 49: 31-54 (2002).

9. Katarzyna Baldy-Chudzik, Neidbach J. and Stosik, M., “REP_PCR fingerprinting as a tool for the analysis of genomic diversity in E.coli strains isolated from an aqueous freshwater environment,” Cellular and Molecular Biology Letters, 8: 793-798 (2003).

10. Knight I. T, Shults S, Kaspar C. W. and Colwell R. R., “Direct detection of Salmonellaa spp. in estuaries by using a DNA probe,” Appl. and Environ. Microbiol., 56: 1059-1066 (1990).

11. Baudart, J., Coallier, J., Laurent, P. and Prevost, M., “Rapid and sensitive enumeration of viable diluted cells of members of the family enterobacteriaceae in freshwater and drinking water,” Appl Environ Microbiol., 68: 5057-5063 (2002).

12. Kapley, A., Lampel, K. and Purohit, H. J., “Thermocycling steps and optimization of multiplex PCR,” Biotechnol. Lett., 22: 1913-1918 (2000).

13. Kapley, A., Lampel, K. and Purohit, H. J., “Rapid detection of Salmonella in water samples by Multiplex PCR,” Water Environ. Res., 73: 461-465 (2001).

14. Purohit H. J. and Kapley, A., “Microbial Quality control of drinking water: PCR as an emerging option,” Trends in Biotechnol., 20: 325-326 (2002).

15. Abramowitz, S., “Towards inexpensive DNA diagnostics,” Trends Biotechnol., 14: 397-401 (1996).

16. Kapley, A., Lampel, K. and Purohit, H. J., “Development of Duplex PCR for Salmonella and Vibrio,” World J Microbiol. & Biotechnol., 16: 457-58 (2000a).

17. Kapley, A and Purohit, H. J., “Detection of etiological agent for cholera by PCR protocol,” Medical Science Monitor, 7: 242-245 (2001a).

18. Bej, A. K., Steffan R. J., DiCesare J, Haff, L. and Atlas R. M., “Detection of coliform bacteria in water by polymerase chain reaction and gene probes,” Appl. Environ. Microbiol., 56: 307-314 (1990).

It is respectfully requested that the claims be amended without prejudice, without admission, without surrender of subject matter, and without any intention of creating any estoppel as to equivalents, as follows.

1. A method for detecting the presence of water born pathogens and indicator microorganism including bacteria from water sample, wherein the said method comprising the steps of:
a) providing water sample and concentrating into suitably sized concentrate of containing target indicator microorganisms in said water sample;
b) isolating the template DNA from target cells obtained from step (a);
c) selecting the target gene carried in said template DNA obtained from step (b) by amplifying the target DNA by using appropriate biotinylated tagged primers and taq DNA polymerase to get desired biotinylated tagged probe containing target gene;
d) hybridizing the biotinylated tagged probe obtained from step (c) with target gene present in the said template DNA followed by enzyme coupled reaction as resulting by change in color wherein blue color indicates the presence of indicator microorganism in the test sample and absence of blue color indicates the absence of said microorganism in the test sample.

2. A method as claimed in claim 1, wherein the water sample is collected from a polluted source selected from any contaminated drinking water.

3. A method as claimed in claim 1, wherein the target cell used are selected from the group consisting of enteric bacteria such as E.coli, Salmonella, Vibrio etc.

4. A method as claimed in claim 1, wherein the said target gene used are selected from the group consisting of lamB gene of E.coli, InvA gene, PhoE gene, SpvA gene and SpvB gene of Salmonella and Ctx gene of Vibrio etc.

5. A method as claimed in claim 1, wherein the primer sequences used for detection of presence of enteric pathogens are selected from the group consisting of:
a) Upper: 5′-CTGATCGAATGGCTGCCAGGCTCC-3′ (SEQ ID NO: 1) and Lower: 5′-CAACCAGACGATAGTTATCACGCA-3 (SEQ ID NO: 2) for detection of E-Coli based on target gene lamB;
b) Upper: 5′-CCTGATCGCACTGAATATCGTACTG-3′(SEQ ID NO: 3) and Lower: 5′-GACCATCACCAATGGTCAGCAGG-3′(SEQ ID NO: 4) for detection of Salmonella based on target gene InvA;
c) Upper: 5′-CTCAGACGGGATTTGTTAGGCACG-3′(SEQ ID NO: 5) and Lower: 5′-GATCTTGGAGCATTCCCACAACC-3′(SEQ ID NO: 6) for detection of Vibrio based on target gene CtxA;
d) Upper: 5′-AGCGCCGCGGTACGGGCGATAAA-3′(SEQ ED NO: 7) and Lower: 5′-ATCATCGTCATTAATGCCTAACGT-3′(SEQ ID NO: 8 for detection of Salmonella based on target gene phoE;
e) Upper: 5′-TGTATGTTGATACTAAATCC-3′(SEQ ID NO: 9) and Lower: 5′-CTGTCATGCAGTAACCAG-3′(SEQ ID NO: 10) for detection of Salmonella based on target gene spvA;
f) Upper: 5′-ATGAATATGAATCAGACCACC-3′(SEQ ID NO: 11) and Lower: 5′-GGCGTATAGTCGGCGGTTTTC-3′(SEQ ID NO: 12) for detection of Salmonella based on target gene spvB;

6. A method as claimed in claim 1, wherein the template DNA is isolated from target cell by dipping the disc containing said cells in 0.5N NaOH solution for 2-5 minutes followed by similar treatment using Tris solution wherein the ratio of NaOH solution and Tris solution used 1:1.

7. A method as claimed in claim 1, wherein the template DNA used is isolated from commercially available strain Escherichia strain ATCC 35150.

8. A method as claimed in claim 1, wherein the target gene used is lamB gene, present in all species of the genera Escherichia.

9. A method as claimed in claim 1, wherein the primer sequence used is consist of all or a substantial part of 5′-CTGATCGAATGGCTGCCAGGCTCC-3′(SEQ ID NO: 1) and 5′-CAACCAGACGATAGTTATCACGCA-3′(SEQ ID NO: 2).

10. A method as claimed in claim 1, wherein the generated biotinylated tagged probe has a size of approximately 309 bp.

11. A method as claimed in claim 1, wherein the pre-hybridazation is carried out with hybridization buffer for 15 minutes at room temperature followed by hybridization wherein denatured lamB probe in sterilized distilled water is added in hybridization buffer.

12. A method as claimed in claim 1, wherein the denaturati on of lamB probe as claimed in claim 11 is carried out at about 95 degree C. for 5-6 minutes.

13. A method as claimed in claim 1, wherein non-specific signals is blocked by using blocking solution of BSA in TBS at about 55 degree C. for 10-15 minutes

14. A method as claimed in claim 1, wherein the signal is generated by diluting Streptavidin-Alkaline phosphatase in the ratio ranging from 1:2000 fold in BSA in TBS at room temperature with biotinylated-hybridized probe.

15. A method as claimed in claim 1, wherein excess and non-specifically bound Streptavidin-Alkaline phosphatase is removed by washing with Tris-NaCl SDS buffer at pH-8.0.

16. A method as claimed in claim 1, wherein signal amplification has been devised with substrate and amplifier for Streptavidin-Alkaline phosphatase as a powder is freshly mixed separately with dilution buffer.

17. A method as claimed in claim 1, wherein the substrate used is reduced nicotinamide adenine dinucleotide phosphate.

18. A method as claimed in claim 1, wherein the amplifiers used are selected from the group consisting of alcohol dehydrogenase and diaphorase

19. A method as claimed in claim 1, wherein the blue color is developed which indicates the presence of water born pathogens and indicator microorganism including bacteria wherein the said color is stabilized by using KOH and acetone in the ratio 5:3.

20. A method as claimed in claim 1, wherein an enzyme-coupled reaction with hybridization increases the sensitivity of the detection protocol.
